(19) United States
(12) Patent Application Publication
(10) Pub. No.: US 2016/0374865 A1
(43) Pub. Date: Dec. 29, 2016

(54) BREAST COVER FOR IMPROVING PUBLIC HEALTH AND PERSONAL HYGIENE IN THE RETAIL APPAREL INDUSTRY

(71) Applicant: Walter Lee Williams, Walton, KY (US)

(72) Inventor: Walter Lee Williams, Walton, KY (US)

(21) Appl. No.: 15/192,498

(22) Filed: Jun. 24, 2016

Related U.S. Application Data
(60) Provisional application No. 62/183,807, filed on Jun. 24, 2015.

(51) Int. Cl. A61F 13/14; A41D 13/05; B65D 85/18; A41D 27/12

(52) U.S. Cl. CPC A61F 13/141; A41D 27/12; A41D 13/0518; A41D 13/0556; B65D 85/18

(57) ABSTRACT

Protective breast covers are provided for improving public health and personal hygiene, particularly in the retail apparel industry. The protective covers preferably feature an impermeable layer to protect the user (and/or the garment) and an adhesive layer to secure the protective cover to the breast. Also provided are methods for improving public health and personal hygiene. Kits including the covers are also provided herein.

### CROSS-REFERENCE TO RELATED APPLICATION

This claims benefit of U.S. Provisional Patent Application No. 62/183,807, filed Jun. 24, 2015, the entirety of which is incorporated by reference herein.

### BACKGROUND

Field of the Invention

This relates generally to devices for improving public health practices for the retail apparel industry. More particularly this relates to personal hygiene devices for covering and protecting breasts or nipples (particularly in women), while also protecting garments from becoming soiled, as well as methods for their use.

Description of Related Art

In the retail apparel industry, it is very common for retail stores to provide dressing rooms or changing rooms that allow shoppers and prospective customers to ‘try on’ clothing, including clothing that comes into direct and intimate contact with the body.

It is important for both the health of shoppers and customers, as well as the public, and also for the protection of the garments (which can become soiled or contaminated) and the industry as a whole that the practices of trying on or determining the fit of garments is done in a sanitary and hygienic manner.

While many states have laws that limit the return of items such as underwear and swimwear to help protect the public health and minimize the spread of diseases via the use and subsequent return of such items, most stores will allow customers to try on items in the store.

For women in particular, there is risk of communicating or contracting diseases of the genitalia. One area that has received little attention and which remains an area of needed public health concern is the breast and particularly the nipples.

For examples, it is known that breasts are subject to infections, such as by Staphylococcus aureus, which may be transmitted through cracks and other breaks in the skin of the breast and particularly of the nipples. Nursing mothers are particularly susceptible to such infection as their nipples may be likely to provide opportunity for the bacteria to enter. Moreover, a woman who has such an infection is more likely to shed/spread the infecting bacteria.

In addition to the risk of infections, there are also a number of conditions are not themselves contagious, but which can create risks of exposure to bodily fluids of another, such as blood, milk, or other discharged fluid for members of the public. Such bodily fluids may potentially be involved in the spread of diseases, pathogens, infectious agents, or the like. For example, there are a number of well-known conditions, including certain cancerous or precancerous conditions that may result in bleeding or other discharges from the nipples.

Even in the absence of frank blood, undesirable discharges or emissions may find their way onto clothing that is being tried on. This is of greatest concern for sales of bras, swimsuits, lingerie, and other items of clothing that make direct contact with a woman's unprotected breasts. Even normal lactation can result in leakage of bodily fluids including colostrum or milk, which may contain a wide variety of viruses and cells (including both bacterial and human white or red blood cells).

Additionally, in the current cultural environment, tattoos on the breast and piercings in the nipple area can become inflamed or infected, also resulting in discharge of bodily material that is a public health concern.

Finally, in addition to communicable diseases, certain chemical compounds, drugs, or even allergens could be transmitted from person to garment to a subsequent person. The risk here could be to the woman, or if a nursing mother, to one or more children who subsequently nurse on a contaminated nipple. Compounds such as illegal or prescription drugs could be pose severe risks to infants via contaminated nipple surfaces, or via local contamination to milk. Exposure to allergens in this manner may provide unwanted, undocumented, and unexplained health risks to either a woman, or a nursing child.

Various protective or absorptive pads and such are available for specific applications. For example, absorptive pads for nursing mothers are available. Such pads are designed to tuck inside a bra and intended to prevent embarrassment by absorbing milk fluids that leak from the mother when her baby is not actively nursing. These absorbent pads are thick to absorb leakage over relatively long periods (e.g. during a nursing mom's work day)

There are also protective pads designed to prevent nipple chaffing particularly in endurance athletes, such as long-distance runners. During running exercises nipples are subject to constant rubbing against any shirt worn, however, women's nipples are often protected by an intervening layer, e.g., a sports bra. As a result, these products are designed primarily for men. The products therefore feature adhesive attachment allowing application to men's nipples. The products are protective but permeable—allowing them to breathe and pass sweat and presumably other fluids. Thus, they are not useful for personal hygiene or public health

Thus, although several types of breast pads are available for different purposes, none are suited for or adaptable for use in improving public heath concerns and personal hygiene (e.g. by preventing exposure to or transmission of etiologic agents) in the retail clothing industry. Moreover, none are suitable for also protecting the garments from becoming soiled or contaminated during a fitting with pathogens, chemical compounds, or the like.

There is therefore a need for new protective devices that are useful for a variety of purposes and provide new features and benefits for consumers and the apparel industry, including personal hygiene as well as the general public health.

### SUMMARY

The inventor has observed a public health and personal hygiene concern prevalent in the retail apparel business. Some issues of public health in the area of retail sales and shopping in the apparel industry have been recognized, and are the subject of precautionary or restrictive measures under the laws of various states. For example, some states have law in connection with the retail sales of bathing suit bottoms for either sex, and for underwear (typically ‘undershorts’ for boys and ‘panties’ for females). There are also some measures regarding the retail sales of footwear. However, the inventor is not aware of any public health protective measure for the widespread practice of ‘trying on’, ‘sizing’, or ‘fitting’ of garments that come into direct contact with nipples, or more generally breasts, e.g. in women. Notwithstanding the discussion regarding public health risks in the foregoing section, women routinely try on bras, lingerie, bathing suit tops, sleepwear, and such with the unprotected breasts (and this nipples) exposed to a garment that has potentially been tried on by other females and thus is very unsanitary and at the very least potentially a risk.

Thus, in a first of the several aspects of this disclosure, the inventor has provided protective coverings to improve public health and/or personal hygiene in connection with the retail apparel industry. The protective covers comprise an impermeable outer layer, which covers at least a portion of a breast (preferably a female breast, and in particular, the nipple portion of the breast) and an inner adhesive layer that allows the protective covering to be removably adhered to and temporarily retained on the breast, while in a retail apparel environment or e.g. while trying on clothes or during a garment fitting. The garments are typically garments that would normally come into direct and intimate contact with a woman's otherwise uncovered breasts. The adhesive layer can preferably be applied to and removed from the skin without removing the epidermis of the area to which it is applied.

Preferably, the outer layer is impermeable to body fluids comprising one or more of sweat, blood, colostrum, milk, pus, white blood cells, bacteria cells, viruses, or other infectious agents, in addition to noxious chemical agents such as legal or illegal drugs, toxins, and allergens.

In another of its several aspects, this disclosure provides packages that generally contain a pair of protective coverings detailed above. In certain embodiments, the packages further comprise a means for disposing the protective coverings after use. The disposal means may be the package itself, or a separate wrapper, bag, or the like to ensure the coverings are disposed of in a reasonably sanitary and aesthetically acceptable manner.

In further aspects of the disclosure, methods of using the protective coverings for improving personal hygiene and/or protecting the public health are provided herein.

Methods are thus provided for enhancing personal hygiene and public health in a setting for trying on or determining the fit of one or more garments. The methods generally comprise the steps of:

obtaining one or more protective coverings as disclosed herein;

applying the protective coverings in a setting for trying on or determining the fit of one or more garments;

maintaining the protective coverings in place during the process of trying on or determining the fit of one or more garments;

removing the protective coverings thereafter, and

disposing of the protective coverings.

The methods are generally suitable for any setting, such as a retail apparel store, where garments are likely to come into contact with an otherwise uncovered breast, preferably a woman's breast, particularly the nipple area.

Also provided herein are methods for enhancing public health and sanitation in a retail apparel setting where shoppers are permitted to ‘try on’ or determine the fit of one or more garments that can come in direct contact with a woman's breasts. The methods generally comprise the steps of

providing a retail apparel setting;

providing one or more fitting areas where shoppers therein are permitted to try on or determine the fit of one or more garments that can come in direct contact with a woman's breasts;

providing one or more protective coverings as disclosed herein in the one or more areas;

instructing shoppers regarding use of the protective covering including instructions to apply the protective coverings to the breast or the garment prior to trying on or determining the fit of one or more garments, and to maintain the protective coverings in place until the process of trying on or determining the fit of the one or more garments is complete;

instructing shoppers to remove and dispose of the protective coverings thereafter, and

providing means for disposing of the protective coverings.

Preferably, the retail apparel store offers bras, bathing suits, halter tops, lingerie, pajamas, sleepwear, robes, or the like that are likely to come into direct contact with a woman's otherwise uncovered breasts.

In yet another aspect, the invention provides novel kits for improving public health in a retail apparel setting, said kit comprising:

a) protective coverings according to this disclosure;

b) instructions for the use, application, and/or disposal of the protective coverings; and

c) a CD, DVD, or links to one or more Internet locations containing additional instructional material including but not limited a manual for use or video instruction for using the protective coverings for public health and/or personal hygiene.

These and/or further aspects, features, and advantages of the present invention will become apparent to those skilled in the art in view of this disclosure.

### BRIEF DESCRIPTION OF THE DRAWINGS

FIG. 1 depicts an embodiment of a protective covering in accordance with the disclosure showing an impermeable outer layer and an adhesive inner layer.

FIG. 2 depicts a view of an embodiment of a protective covering showing an additional layer between the outer layer and the adhesive layer. It can be seen the adhesive layer need not completely cover any adjacent layer, but rather can be present as a ring or even strategically placed amounts of adhesive such that the protective can be both held in place and easily removed. The additional lay can provide a comfortable surface for contact with sensitive skin and may contain supplements or additives to soothe, treat, or condition the skin, e.g. of the nipple.

FIG. 3 shows various packages including one or more breast covers in accordance with the disclosure.

FIG. 4 provides a flowchart of an embodiment of a method for use in improving personal hygiene in e.g., retail apparel settings.

FIG. 5 is a flowchart of an embodiment of a method for improving public health in e.g., retail apparel settings.

### DETAILED DESCRIPTION

Provided herein are protective covers for improving public health with respect to the retail apparel industry, particularly for women's bras, lingerie, pajamas and other sleepwear, and other tops. Also provided are packages containing the covers in pairs for convenience and personal hygiene, as well as methods of improving public health or increasing personal hygiene via the use of such protective covers.

The inventor has surprisingly discovered a significant public health and personal hygiene issue in the retail apparel industry and similar settings that has heretofore apparently eluded public health officials. Moreover, the inventor has developed simple structure adapted for resolving the problem that is comfortable, useful, convenient to use, and inexpensive, particularly relative to the costs associated with the potential risk inherent in the current environment.

In operation, the protective coverings are primarily designed to cover women's breasts, particularly the nipple portion of the breasts, while trying on or fitting garments that would otherwise come into direct, intimate contact with the breast. In certain embodiments, the covers are applied to the garments directly in the area that would otherwise contact the breast, rather than applied to the breast themselves. The protective coverings are adapted for public health and provide significant personal hygiene benefits in certain settings where clothing is tried on, fitted, or the like, e.g. in retail apparel stores by shoppers prior to purchase. In various applications, the coverings are positioned against the user's breasts (particularly, the nipples).

The protective covers can be packaged in pairs for convenience and methods are provided for use of the covers to improve public health and/or protect personal hygiene.

Definitions & Abbreviations

Unless expressly defined otherwise, all technical and scientific terms, terms of art, and acronyms used herein have the meanings commonly understood by one of ordinary skill in the art in the field(s) of the invention, or in the field(s) where the term is used. In accordance with this description, the following abbreviations and definitions apply.

As used herein, the singular form of a word includes the plural, and vice versa, unless the context clearly dictates otherwise. Thus, the references “a”, “an”, and “the” are generally inclusive of the plurals of the respective terms. For example, reference to “a breast” or “a garment” includes a plurality of such “breasts” or “garments.”

The words “comprise”, “comprises”, and “comprising” are to be interpreted inclusively rather than exclusively. Likewise the terms “include”, “including” and “or” should all be construed to be inclusive, unless such a construction is clearly prohibited from the context. Further, forms of the terms “comprising” or “including” are intended to include embodiments encompassed by the phrases “consisting essentially of” and “consisting of”. Similarly, the phrase “consisting essentially of” is intended to include embodiments encompassed by the phrase “consisting of”.

As used herein, “adhesive” or “an adhesive” means any composition or material that is able to allow a protective to be removably attached as defined herein. In some embodiments, an adhesive layer is a layer of material that has the ability to have the impermeable layer to adhere to, remain in contact with, or stick to the skin of the wearer and/or a portion of the garment for a duration and with enough sufficient intensity to allow a garment to be tried on or fitted including the removal of the garment with dislodging or allowing the breast or nipple area to come into intimate or direct contact with the garment during the process. Accordingly, adhesive includes glues and the like but also includes friction, cohesive forces, thin film forces involving skin, moisture, and the like, which may be properties of a particular material. This in certain embodiments contemplated for use herein, the impermeable layer may itself have sufficient ability to adhere to the skin of a wearer, and/or to adhere to a garment to satisfy the requirement for adhesive properties herein.

As used herein “bodily fluids” means any fluid produced by, secreted by, emitted from, or located on a body. Bodily fluids can comprise any one or more of sweat, blood, colostrum, milk, pus, white blood cells, bacteria cells, viruses, or other infectious agents (including prions), allergens, or chemical compounds that are secreted from or applied to the breast. Chemical compounds including both illegal and legal drugs, as well as compounds that may be naturally found, and by-products or metabolites of any of the foregoing.

As used herein, “breast” refers to a mammalian mammary gland and to the corresponding areas of the male chest. While certain applications of the protective coverings may pertain to other mammals, generally, breast means the human breast, including the nipple. The protective coverings in various embodiments described herein are intended to cover a “portion of the breast”, which can refer to any portion on the breast whether or not it includes “the nipple area.” In certain embodiments, the protective covers are described as covering “the nipple area”.

As used herein “nipple” means the nipple and the surrounding areola area. Breasts come in various sizes, and similarly, the size of the nipple and areola will vary from person to person. Thus, “the nipple area” as used herein means in general an area that encompasses at least a substantial part of the nipple and areola (in e.g., a woman with very large nipples and areola) to an area that encompasses the entire nipple, areola, and a surrounding portion of the breast (in e.g., a woman with very small nipples and areola).

As used herein “removably attached” or the like, with reference to the protective covers, means that the cover can be removed from the breast or the nipple area without substantially damaging or irritating the surrounding skin. While the inventor intends that there be no significant pain during the removal of the protective covers, especially from the sensitive nipple area, pain tolerance in subjective and given the sensitivity of the nipple and surrounding area, it is impossible to ensure that removal is completely painless to all. Nonetheless, the adhesive used in the application is selected so as to minimize any significant risk of damage or pain during removal. While the protective covers are intended to remain in place during fitting and subsequent removal of clothing items, the need for the protective cover is very short term. Thus, unlike adhesive bandages or transdermal patches, which must remain in place for extended periods and thus require adhesives with suitable properties for that purpose, the protective covers of this disclosure comprise only sufficient adhesive for relatively short-term use. Any such adhesive preferably allows for removal consistent with the foregoing—i.e. without substantial damage or pain to the wearer. Preferably the covers can be removed without any pain or discomfort to a vast majority of the users, and without and degree of damage (including redness, irritation, chafing, or the like) to the skin on which the adhesive is applied and removed. In certain embodiments, the protective covers may be removably attached to the garment, in which case the garment is not damaged and there are no visible signs of wear as a result of the removal of the protective cover from the garment after e.g. a fitting.

As used herein “substantial” or “substantially” mean a significant part of something. In various embodiments, a majority of the thing is intended, i.e. more than half (e.g. >50%). In other cases, the amount is greater than two-thirds, three-quarter, seven eighths or even more. In certain embodiments, the percentage may be more than 60%, 70%, 80%, or even 90% or 95%. “Substantially all” means almost the entire thing, such as 75%, 85% or more.

As used herein “impermeable” means not allowing something to pass through. When used to describe a layer herein, it means that that layer will not allow the specified thing (generally a fluid) to pass through. Unless otherwise specified in a particular context, impermeability with respect to a layer is two-way. I.e., if a layer is impermeable to e.g. a fluid, such as blood or milk, that fluid cannot pass from the breast (or nipple) to the garment, nor could any trace or that material pass from the garment to the breast (or nipple). More broadly, ‘impermeable’ herein also generally means ‘impervious’ to a particular substance, i.e. not allowing that substance to pass through whether or not it is a fluid. In the context here, impermeability does not presuppose a particular mechanism —i.e. impermeability can result from any property of the layer including the mean pore size, pore structure, surface charges, net charges or other properties, or combinations of one or more of the foregoing that may prevent one material from passing through another.

Where used herein, ranges are provided in shorthand, so as to avoid having to list and describe each and every value within the range. Any appropriate value within the range can be selected, where appropriate, as the upper value, lower value, or the terminus of the range.

The methods and devices and/or other advances disclosed here are not limited to particular methodology, protocols, and/or structures described herein because, as the skilled artisan will appreciate, they may vary. Further, the terminology used herein is for the purpose of describing particular embodiments only, and is not intended to, and does not, limit the scope of that which is disclosed or claimed.

Although any devices, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent permitted under applicable law. Any discussion of those references is intended merely to summarize the assertions made therein. No admission is made that any such patents, patent applications, publications or references are prior art, or that any portion thereof is either relevant or material to the patentability of what is claimed herein. Applicant specifically reserves the right to challenge the accuracy and pertinence of any assertion that such patents, patent applications, publications, and other references are prior art, or are relevant, and/or material.

Abbreviations

The following abbreviations apply unless indicated otherwise:

cm: centimeter(s);

in: inches; and

sq. square.

### DETAILED DESCRIPTION OF ILLUSTRATIVE EMBODIMENTS

In a first of its several aspects, this disclosure provides protective covers for protecting transmission of fluids and other material from a garment to a woman's breast and vice versa. Thus, the covers protect the garments from becoming contaminated or soiled, while simultaneously protecting a woman from exposure to communicable agents or noxious substances present on a garment. The protective covers in various embodiments comprise an impermeable outer layer that covers at least a portion of a woman's breast; preferably the nipple area is covered or substantially covered. In some embodiments, the protective covers may alternatively be applied directly to the garment, e.g. to the portion of the garment that would contact the breast, particularly the nipple area. An inner adhesive layer allows the protective covering to be removably adhered to and retained on the breast (or garment, as the case may be) while in a retail apparel environment or during a garment fitting.

In presently preferred embodiments, the impermeable outer layer covers at least the nipple portion of the breast. Generally, the adhesive layer can be applied to and removed from the skin without removing the epidermis of the area to which it is applied. Moreover, the epidermis of the area to which the protective covers are attached is preferably not substantially pulled, stretched, reddened, or irritated. Preferably the removal is easily affected by hand, and does not cause pain to the person wearing the cover.

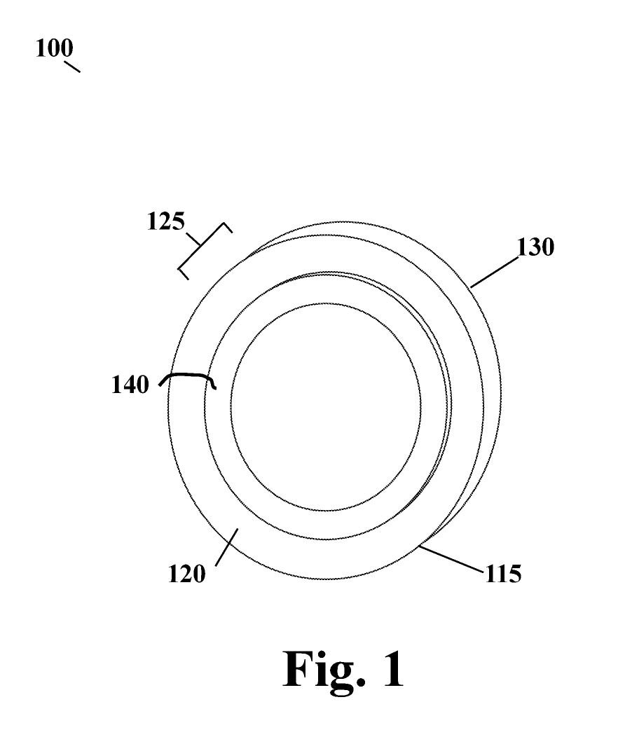

The protective covers can be better understood by reference to the figures. FIG. 1 depicts an embodiment of the protective cover in a simple form. The cover 100 features an outer impermeable layer 120, and an inner adhesive layer 140. The outer layer 120 can be any shape desired. The overall diameter of the cover (for a circular cover) can range from about 1 inch or less, to 2 inches, 3 inches, or even more as may be useful. Preferred protective covers of circular design are thus about 1 to 3.5 inches in diameter in certain embodiments, 1.5 to 3 inches in diameter in other embodiments, 1.8 to 2.8 inches in diameter in other embodiments.

It is to be understood that the overall size selected preferably comprises an coverage area sufficient to cover an area that comprises at least substantially all of the nipple area (as defined herein) (e.g., on a person with a larger nipple area), to an area comprising the nipple area plus a portion of the breast (e.g., on a person with a smaller nipple area). In various embodiments, the coverage area comprises less than about 10 sq. inches. In other embodiments, the covers cover an area less than about 9, about 8, about 7, about 6, or about 5 sq. inches. In yet other embodiments, the coverage area is less than about 5, or about 4 sq. inches, or less than about 3.5 sq. inches.

The covers may have a outside profile 125 of any shape such as simple such as circular, oval, elliptical, or simple polygonal (e.g. square, rectangular, pentagonal, etc.) and they may be more complex with outside profile that are star-shaped, cloud shaped. The shapes may be selected to inspire or contribute a fun or pleasant feel to the covers—limited only by the designer's imagination. Shapes such as lips, eyes, or the like may optionally be complemented with coloring, printing, patterns or designs on the outer aspect or outer surface 130 of the outer layer 120. Such colors or shapes may be varied, and may contain a logo, words, or similar material. The covers may additionally be lightly scented.

It will be understood by the skilled practitioner that any coloration, printing, scents, or the like, must not transfer to the garment so as to cause problems. The covers may also optionally comprise additional adornment (not shown) or decoration provided that any such adornment (1) not interfere with the fit of the garment; (2) not alter, damage, or otherwise adversely affect the garment, and (3) not cause any discomfort or interference with the wearing or removal of the protective covers. Such adornment can be conveniently attached to the outer layer 120 or elsewhere as may be useful.

With further reference to FIG. 1, the inner adhesive layer 140 is shown as a ring of adhesive applied to the inner aspect or inner surface 115 of the outer layer 120. The adhesive layer 140 may be present as a complete area applied to the entirety of the inner aspect 115 of the outer layer 120 (or to the inner aspect of any optional layer that is attached to or adjacent to the inner aspect 115). Alternatively, in certain embodiments, the adhesive layer 140 may comprise adhesive on any portion or portions or such inner aspect provided that the adhesive layer contain sufficient adhesive to retain the cover where positioned on the breast (such as over the nipple area).

In various embodiments, adhesive may be applied in lines, arcs, spots, dots, or the like, along the inner aspect of the innermost layer of the protective cover. In one embodiment, the adhesive is preferably applied such that the adhesive does not make contact with the most sensitive areas of the breast, i.e. the nipple area. Instead, the adhesive layer provides adhesive to areas outside of the nipple area, and secures the cover the breast in the manner. This minimizes any risk of discomfort of sensitivity when applying or removing the protective cover from the breast.

In certain embodiments, prior to use of the cover, the adhesive layer 140 may be attached to a temporary backing layer (not shown). The backing layer comprises any material whose surface properties and the like allow it to easily stick to and be removed from the adhesive layer. The end user would remove the backing layer at the time of application of the protective cover to the breast. Such backing layers are familiar to the skilled artisan and examples of analogous backing layers can be found on a wide variety of common consumer goods, including adhesive bandages, stickers (including adhesive devices for sticking to human skin).

In certain embodiments, the outer layer 120 of the protective covering is impermeable to body fluids. The outer layer can prevent or substantially prevent any of sweat, blood, colostrum, milk, pus, white blood cells, bacteria cells, viruses, or other infectious agents, and allergens, as well as chemical agents including legal and illegal drugs, from passing through from a person wearing the cover to the garment, and can also prevent any such materials from passing off a contaminated garment to the person.

In certain embodiments, the protective coverings provided herein are very thin. They preferably have an overall thickness that does not substantially change or interfere with the fit of a garment worn over the protective covering. In various embodiments, the overall thickness is less than about 5 mm, and more preferably, less than about 3 mm, less than about 2.5 mm, or even less than about 2 mm. Even thinner covers are contemplated herein such as covers with thickness of about 1.5, or about 1, or less.

As new materials become available, thinner covers may be useful. Moreover, materials may be developed, discovered, or determined to have all of the properties useful herein such that an impermeable layer and a layer that is sufficiently adhesive for the purposes required to create a single layer protective cover comprising a single material, a composite, hybrid, or the like. Such embodiments are also contemplated for use herein, particularly with the methods provided herein below.

In some respects the protective covers disclosed herein have similarities to e.g. adhesive bandages, however the skilled artisan will instantly appreciate the significant differences including the strength of the adhesive (as required for the amount of time such bandages are intended to remain in place) and the impermeability of the outer layer. Adhesive bandages typically have an outer layer that is fairly permeable. In fact in common designs the outer layer is actually perforated to allow both air and moisture to pass through to help promote healing of the underlying wound which could become problematic in an environment that is wet and potentially anaerobic. Moreover, although an absorptive layer is typically present on adhesive bandages, anyone who has ever worn such a device knows that they can readily ‘bleed through’ and will need to be changed.

In contrast, the protective covers provided herein have an adhesive layer that provides much less grip through either the net amount of adhesive present, or the type of adhesive used, or a combination thereof. Such adhesive layer is more than adequate for the intended use of the protective covers but would not function well for an adhesive bandage. Moreover, even when the covers include an absorptive layer (as described below) the outer layer is impermeable. The covers will not ‘bleed-through’ and there is no need for them to allow air or moisture to pass.

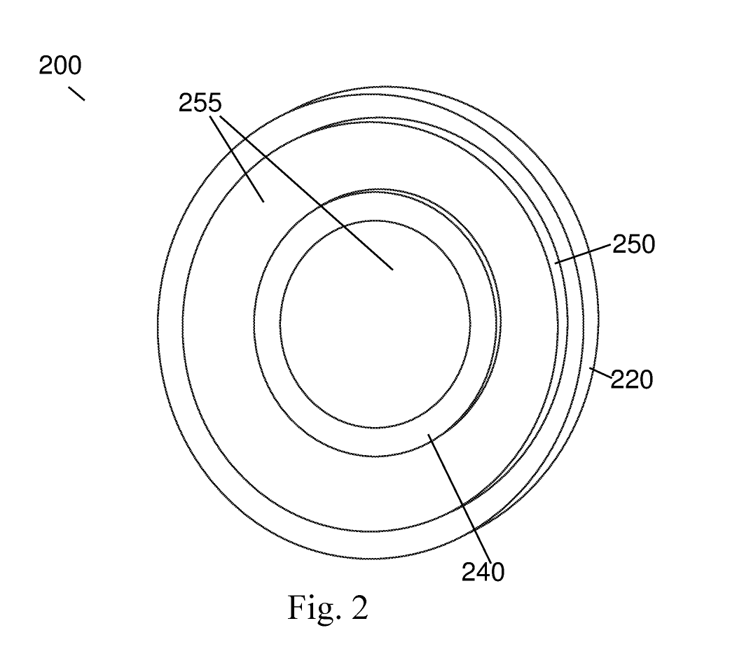

Thus, in other embodiments, the protective coverings further comprise one or more intermediate layers interposed between the outer layer and the inner layer, each such intermediate layer providing one or more functional properties for the covering in terms of comfort or protection. With further reference to the figures, FIG. 2 shows an embodiment 200 comprising an additional absorptive layer 250 disposed between the outer layer 240 and the inner adhesive layer 220. As can be seen, the vast majority of the inner aspect 255 of the absorptive layer 250 is exposed to the skin (e.g. the nipple area) of the person wearing the cover. As discussed below, the adhesive layer 220 preferably does not directly contact the nipple but rather only contacts surrounding areas. The intermediate layer(s) generally (e.g. absorptive layer 250 in FIG. 2) can comprise one or more of a comfort layer, an absorptive layer, or a carrier layer; and the functional properties comprise one or more of comfort during wear or removal of the protective covering, the ability to absorb one or more of sweat, blood, colostrum, milk, pus, white blood cells, bacteria cells, viruses, or other infectious agents, allergens, or chemical agents (including legal and illegal drugs), the ability to carry one or more useful compounds comprising a skin emollient, an antibiotic, a biocidal or virucidal compound, an oil or cream, a vitamin or a derivative or analog thereof, an herbal extract, or a scent, or the like.

In some embodiments where useful compounds such as emollients are used, they are preferably applied to the absorptive layer or the inner aspect of the absorptive layer to maximize the contact with the skin. With further reference to FIG. 2, the inner aspect 255 of the absorptive layer 250 can be coated, impregnated, surface-treated, or the like with compounds 260 that will contact the skin and enhance the experience and comfort of the user, provide additional sanitary benefits, or the like.

In certain embodiments, the absorptive layer may not extend under or to the adhesive layer. In other words, in such embodiments, the adhesive layer does not directly contact the nipple area, whereas the absorptive layer will preferably contact the nipple area so as it maximize the absorptive/protective function(s) to that area, as well as to the garment itself.

The user can easily accomplish removal of the protective covers by taking one peripheral edge of a cover and gently pulling the cover off of the skin. Although the adhesive qualities of the cover are sufficient for holding the cover in place for short-tem use, but not as strong as adhesives that might be required for e.g. long term retention of other devices, removal does not require much force, however because the cover is on or near highly sensitive tissue, facilitating removal by making it faster, easier, and less prone to accidental pinching of e.g. skin, is desirable. Accordingly, in certain embodiments, the protective covers further comprise means for more conveniently removing the protective covering. In other embodiments, the removal means comprises a tab, a tape, a string, a modified edge or corner, or the like that will provide a convenient hold on the cover for removal. In yet other embodiments, a raised portion or segment of the outer layer, or connected to the outer layer, can conveniently provide a hold on the cover for removal.

Although the foregoing disclosure primarily describes the protective covers as being adhered to the breast, it is also contemplated herein the such protective covers can be adhered directly to the garment prior to trying on or fitting such garments. For such embodiments, the overall construction of the protective cover can be substantially similar as described above. Thus, in the most basic embodiments, the adhesive layer is attached to the impermeable layer in an identical fashion to that described above. The adhesive layer is then used to retain the protective cover on the garment, rather than on the breast. The adhesive used in such embodiments still preferably has properties that allow it to remain in place for a short period of time, e.g. and to withstand handling expected during the trying on or fitting of the garment. The adhesive should be capable of being readily removed without ‘damaging’ or substantially damaging the garment in any noticeable or functional manner. The skilled artisan will then appreciate that such embodiments can be used in a “dual-function” mode wherein the user can decide whether to apply the protective cover to the garment, or to her breast. In either mode, the protective cover will provide the desired benefits of improving public health and personal hygiene since there will be no direct, intimate contact between the garment and the breast in the area protected by the cover(s).

The skilled artisan will appreciate however, that the adhesive layer in embodiments involving additional layers such as an absorbent layer or a layer added for comfort, will actually preferably be attached to the outer aspect of the impermeable layer, rather than attached to the inner aspect of the inner most layer relative to the breast. In other words, the adhesive layer will be closest to the garment in such embodiments, while the absorbent layer or comfort layer will still contact the user's breast, e.g. the nipple area. In such embodiments, prior to use the protective covers can employ a backing on the adhesive layer, and for sanitary reasons, may also include a cover or backing that protects the aspects of the protective that will directly contact the breast.

Thus, this disclosure provides protective covers that apply directly to the breast, or alternatively covers that apply to the garment, with each type having advantages and disadvantages for the shopper/prospective buyer and for the retailer. The covers are similar in design with the exception of the possible of whether the adhesive is applied to the innermost aspect or outermost aspect of the protective cover, or whether the cover can function in a dual mode. Embodiments that apply directly to the garment rather than to the breast provide advantages for women that are not comfortable applying the covers to their breasts. However, a new set of such covers may be required for each garment to be tried on, depending on the properties of the cover, particularly the adhesive properties.

In another aspect of the disclosure, provided herein are packages containing one or more protective coverings as described herein.

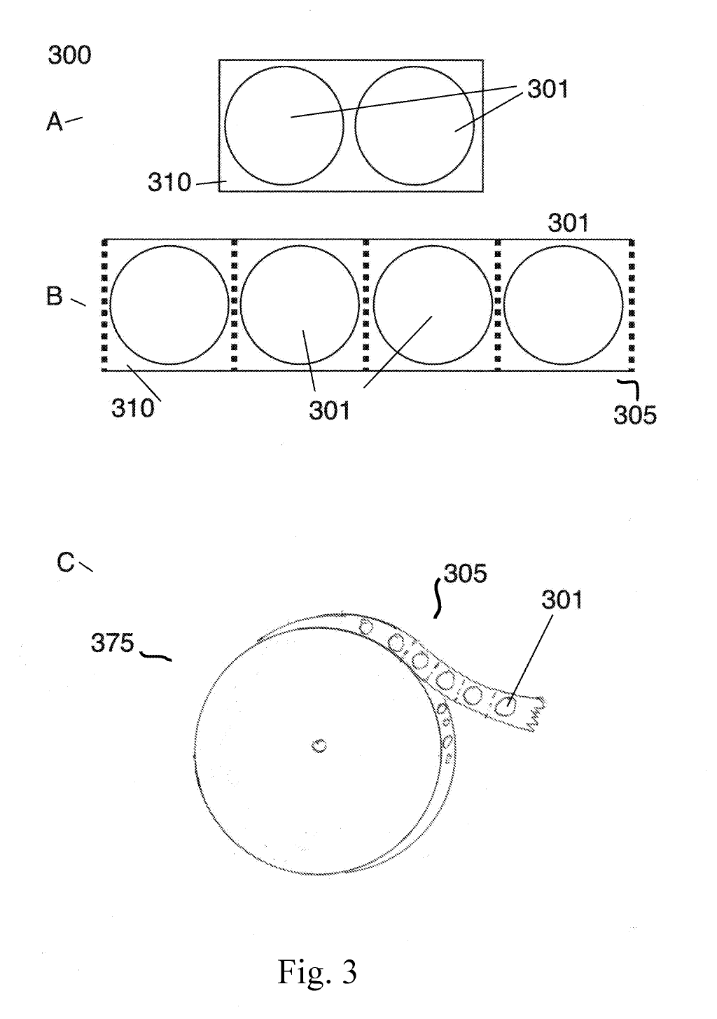

In certain embodiments, the packages comprise a pair of protective breast covers for convenience to the end user. With further reference to the figures, FIG. 3, Panel A depicts a pair of protective covers 301 attached to a single backing 310 in a single package 305. In other embodiments, the packages contain a plurality of protective covers. FIG. 3, Panel B depicts a plurality of individual protective covers 301 on perforated backings 310. The package 305 can comprise any number of such packages and can further include a dispensing box or the like (not shown) for convenience. Because of the adhesive layer, the protective covers may each include a backing as described above. In one embodiment a pair of protective covers is present on a single backing layer. The backing for such embodiments may comprise a flexible layer or a stiffer layer (such as a card or the like) from which the protective covers are ‘peeled’ or removed at the time of use. The packages may comprise dispensing units, such as self-feeding dispensers (like for example facial tissue boxes). Alternatively, dispensing units may be used to contain and dispense other packages contemplated for use herein.

In certain embodiments, the packages may comprise continuous rolls of protective covers providing 50, 100, 200, or even more protective covers. Such rolls may be conveniently placed in retail locations such as in fitting and/or changing rooms where garments are tried on or fitted. In one embodiment, such a roll further comprises an additional package, such a box, which is adapted to allow the roll to turn and feed the protective covers out of the side thereof.

In one embodiment, the roll comprises a continuous common backing for a plurality of protective covers (as with a roll of stickers for a child, or a roll of stickers in a retail store that say ‘PAID’ or such). In such embodiments, the user removes the protective cover from the backing as needed and the now empty backing can be, e.g. torn off thereafter. In one embodiment, the backing may be lightly scored, perforated, or the like to facilitate removal of the backing before or after the protective covers are peeled off for use. In such embodiments, the inner aspect of the inner layers or layers contacting the skin may remain sanitary or potentially even commercially sterile, while the outer aspect of the outer layer is exposed to the environment.

In another embodiment featuring packaging as continuous rolls, the roll may comprise a connected series of individual packages. In such embodiments, the individual packages can comprise individual protective covers, or a pair of protective covers. In such applications, the individual packages can comprise sterile protective covers, as these covers are not exposed to the environment or opened until the user is ready to apply them. An embodiment of a roll 305 of such protective covers is depicted in FIG. 3, Panel C. A plurality of individual covers 301, each on a backing 310 are shown on a continuous roll 310. Each cover in this embodiment is attached to a separate backing 310, however, the protective covers could alternatively be paired for convenience, e.g. as in Panel A. The individual packages 310 can be conveniently separated via the perforations provided between packages 310. The roll can be used on a roll dispenser, or can be placed in a box for storage and easy dispensing.

The skilled artisan will appreciate that packaging items such as the protective covers in pairs has advantages and disadvantages. Packing individually requires more packaging and processing, while packaging in pair provides some efficiency since the covers will generally be used in pairs. However, if there is a problem (e.g. a defective cover, or as a result of a ‘user error’) with a single protective cover packaged as a part of a pair, the full pair will be wasted and a new pair will be used, whereas when packaged separately as individual covers, only one cover would be wasted and only one needed to be replaced.

The packages may comprise any material readily available for such purposes, including paper, coated paper, waxed paper, plastic or polymers of any type, particularly polyester and/or polyethylene and derivatives thereof), cardboard, foil, metalized films, MYLAR, bo-pet, and other multilayered packaging, and the like.

In certain embodiments, the packages further comprise a means for disposing of the protective coverings after use, wherein the means may be the package itself. For example, the package may comprise, e.g. a foil packet that is designed to allow the protective to be return thereto after use, and disposed. In one embodiment, the packages comprise a zipper-type closure for sealing the packet after the used covers are placed therein. In other embodiments, the package can be turned inside out to receive the used covers and then turned outside out when disposing the used covers.

In one embodiment the protective coverings are sanitized, commercially sterile, or even sterile. The skilled artisan will appreciate that such sterilization can be accomplished for example via exposure to cobalt radiation, ethylene oxide gas, biocidal ultraviolet light, plasma, dry heat, or steam depending on the properties of the protective coverings, the packaging, the initial biological load, and the reduction in biological load desired.

In various embodiments, the packages provided herein further comprise instructions or directions for use. For example the instructions may explain how to use the protective covers to obtain personal hygiene benefits in a retail apparel store, or while trying on or determining the fit of one or more garments.

In yet another aspect of the invention, methods for enhancing personal hygiene and public health are provided. The methods are applicable in any setting for trying on or determining the fit of one or more garments. The methods generally comprise the steps of:

obtaining one or more protective coverings according to the instant disclosure;

applying the protective coverings in a setting for trying on or determining the fit of one or more garments;

maintaining the protective coverings in place during the process of trying on or determining the fit of one or more garments;

removing the protective coverings thereafter, and

disposing of the protective coverings.

The setting in one embodiment is a retail apparel store. In other embodiments the setting could be a dressmaker, a seamstress, a clothing designer or the like. The setting could also be a theater, movie production set, television studio, entertainment center/park, cruise ship, or the like where people use costumes or other clothing that is not their own personal clothing, or which is shared amongst multiple persons who may each wear the costume or other garment from time to time.

In one embodiment, the protective covers comprise a single layer, or a single material that provides an impermeable and sufficient adhesive properties to accomplish the protective role for public health and/or personal hygiene with respect to the retail apparel experience including trying on or fitting of garments that contact a woman's breasts.

The applying step can comprise applying directly to the breast or nipple area, or applying to the garment in a portion that would otherwise contact the breast and/or nipple. In one embodiment, the covers designed with layers that are symmetric except for the addition of the adhesive layer—allowing the covers to be readily applied to the breast or to the garment as the user's discretion.

In one embodiment, the protective coverings used in the method are commercially sterile. The step of disposing the protective coverings is preferably done in a sanitary manner, such as by having a separate container to dispose of the used covers, or by have a defined and discreet method of disposal available.

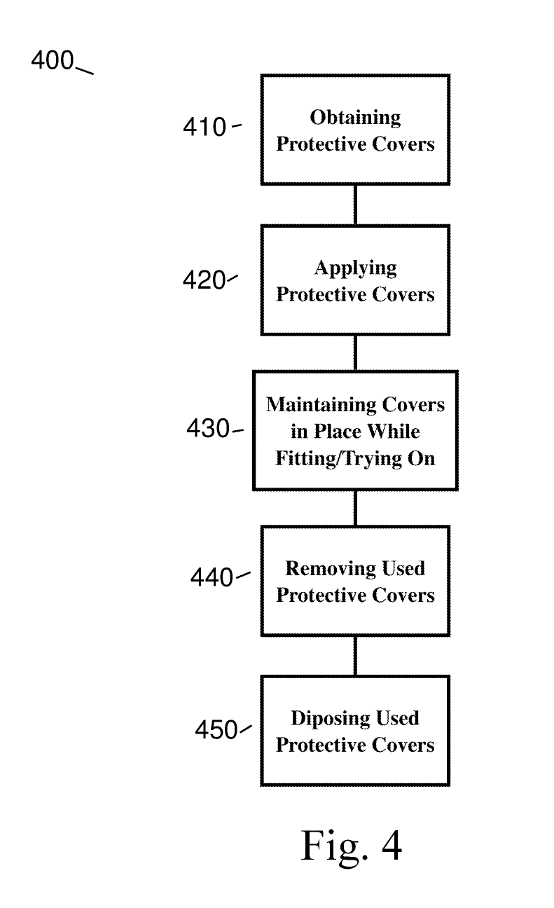

FIG. 4 shows a flow chart of a method according to this aspect of the invention. The skilled artisan will appreciate that the user of the protective covering can obtain the covering via a variety of means, including by purchasing the covers at a retail store such as pharmacy or store carrying personal care items. Alternatively, the clothing store or other setting may sell or provide the protective covers for the convenience of the customers. Preferably, the retail store provides the protective covers in the fitting rooms or dressing areas of the store.

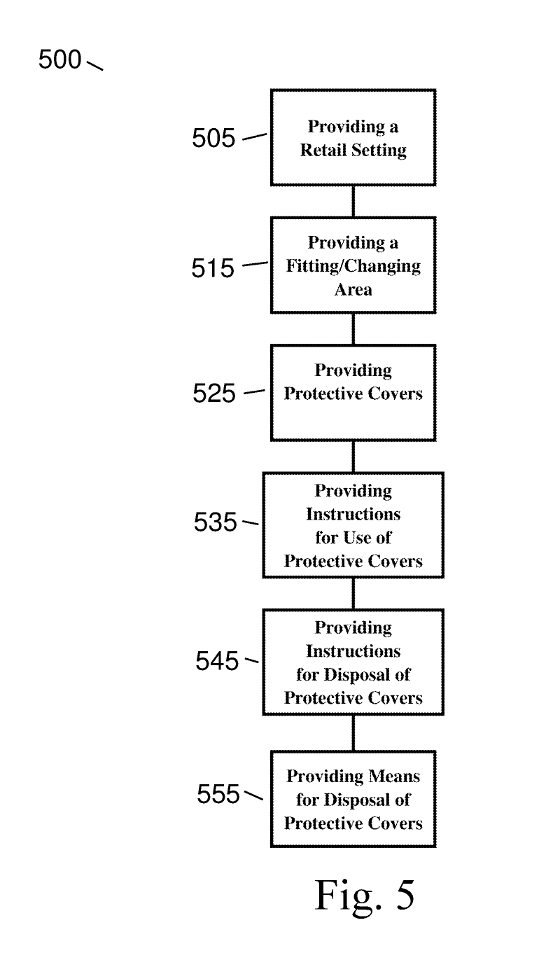

In yet another of its several aspects, this disclosure provides methods for enhancing public health and sanitation in a retail apparel setting where shoppers are permitted to try on or determine the fit of one or more garments that can come in direct contact with a woman's breasts. An embodiment of such method is shown in FIG. 5.

Generally, in such settings, the methods comprise the steps of

providing a retail apparel setting;

providing one or more fitting areas where shoppers therein are permitted to try on or determine the fit of one or more garments that can come in direct contact with a woman's breasts

providing one or more protective coverings according to claim 2 in the one or more areas;

instructing shoppers regarding use of the protective covering including instructions to wear the protective coverings or apply the covers to a garment prior to trying on or determining the fit of one or more garments, and to maintain the protective coverings in place until the process of trying on or determining the fit of the one or more garments is complete;

instructing shoppers to remove and dispose of the protective coverings thereafter, and

providing means for disposing of the protective coverings.

In various embodiments, the retail apparel store sells bras, bathing suits, halter tops, lingerie, pajamas, sleepwear, robes, or the like that are likely to come into direct contact with a woman's breasts.

The protective coverings can be sterile, or only commercially sterile, or at least sanitary prior to use. The step of disposing the protective coverings is done in a sanitary manner—so as to be consistent with the overall function of improving public heath and personal hygiene. As described above, the protective coverings can comprise any covering whose qualities are consistent with the properties described herein (e.g. impermeability and adhesive properties) whether such comprises a single layer or multiple layers, or a single material.

The protective coverings can be provided in bulk (e.g. in boxes or bags), or can be packaged individually, in pairs, or provided in rolls or such.

In various embodiments, the method comprises instructing steps that provide sufficient instructions for use and/or disposal of the protective coverings via written instructions, video, audio, or online presentation, or via oral instructions from a retail clerk.

The instructing steps can thus comprise oral/verbal instructions, instructive signs, photographs, pictures, drawings, or diagrams, or other means of instructing. In one embodiment, the instructions comprise audiovisual materials, such as a video clip, or a narrated series of pictures or diagrams to explain or demonstrate the proper use of the protective covers and/or the benefits of using such covers. In one embodiment, a kiosk providing recorded is provided in the fitting areas. In such embodiments, the kiosk, which can have an attendant or be unattended, can provide dispensers for obtaining the protective covers or packages thereof and instructions for their use. Such a kiosk can be very small and might comprise a tablet computer (on a wall or on a stand) or multimedia player or the like featuring a looped video explanation, and a box or roll of protective covers. The tablet computer could be linked to an Internet site provided by the retailer for promoting the use and benefits of the protective covers in their locations. In one embodiment each fitting room can be adapted to contain such a kiosk. In other embodiments, a fitting area with multiple fitting rooms can be serviced by one or more common kiosks, whether attended or unattended. In some embodiments, one or more signs are posted in the retail store or in the fitting areas providing information promoting public health via the use of the protective covering, or communicating the public health risks of communicating disease from garments without adequate protective measures. Such signs can be used in conjunction with other means for instructing or teaching shoppers about the covers and how to best use them, for example the kiosks described above.

In a final aspect hereof, kits are provided for improving public health in a retail apparel setting. The kits generally provide:

protective coverings according to this disclosure;

instructions for the use, wearing, applying, and/or disposal of the protective coverings; and

a CD, DVD, or links to one or more Internet locations containing additional instructional material including but not limited a manual for use or video instruction for using the protective coverings for public health and/or personal hygiene.

In one embodiment, the kits further comprise one or more of:

a dispenser for use in the fitting areas; said dispenser for dispensing individually or in pairs, the protective coverings to shoppers trying on or determining fit of a garment; and

one or more signs for use in the retail apparel store providing information in the public health risks associated with unprotected trying on or fitting such garments, or the public health benefits of using the protective coverings.

The scope of the invention is set forth in the claims appended hereto, subject, for example, to the limits of language. Although specific terms are employed to describe the invention, those terms are used in a generic and descriptive sense and not for purposes of limitation. Moreover, while certain presently preferred embodiments of the claimed invention have been described herein, those skilled in the art will appreciate that such embodiments are provided by way of example only. In view of the teachings provided herein, certain variations, modifications, and substitutions will occur to those skilled in the art. It is therefore to be understood that the invention may be practiced otherwise than as specifically described, and such ways of practicing the invention are either within the scope of the claims, or equivalent to that which is claimed, and do not depart from the scope and spirit of the invention as claimed.

What is claimed is:

1. A protective covering comprising an impermeable outer layer that covers at least a portion of a woman's breast, and an inner adhesive layer that allows the protective covering to be removably adhered to and retained on the breast while in a retail apparel environment or during a garment fitting; wherein the impermeable outer layer covers at least the nipple portion of the breast, and wherein the adhesive layer can be applied to and removed from the skin without removing the epidermis of the area to which it is applied.

2. The protective covering of claim 1 wherein the outer layer is impermeable to body fluids comprising one or more of sweat, blood, colostrum, milk, pus, white blood cells, bacteria cells, viruses, or other infectious agents, allergens, and chemical agents, including legal and illegal drugs.

3. The protective covering of claim 2 having an overall thickness that does not substantially change or interfere with the fit of a garment worn over the protective covering.

4. The protective covering of claim 2 having an overall thickness of less than 3 mm, less than 2.5 mm, or less than 2 mm.

5. The protective covering of claim 2 further comprising one or more intermediate layers interposed between the outer layer and the inner layer, each such intermediate layer providing one or more functional properties for the covering in terms of comfort or protection.

6. The protective covering of claim 5 where the intermediate layers comprise one or more of a comfort layer, an absorptive layer, or a carrier layer; and the functional properties comprise one or more of comfort during wear or removal of the protective covering, the ability to absorb one or more of sweat, blood, colostrum, milk, pus, white blood cells, bacteria cells, viruses, or other infectious agents, the ability to carry one or more useful compounds comprising a skin emollient, an antibiotic, an oil or cream, a vitamin or a derivative or analog thereof, an herbal extract, or a scent.

7. The protective covering of claim 2 further comprising means for conveniently removing the protective covering, said means comprising a tab, a tape, a string, a modified edge or corner, or a raised portion or segment.

8. A package containing a pair of protective coverings according to claim 2.

9. The package of claim 8, further comprising a means for disposing of the protective coverings after use, wherein the means may be the package itself.

10. The package of claim 8 wherein the protective coverings are individually wrapped, or wrapped as a pair.

11. The package of claim 8 wherein the protective coverings are commercially sterile.

12. The package of claim 8 further comprising directions for use thereof to obtain personal hygiene benefits in a retail apparel store, or while trying on or determining the fit of one or more garments.

13. A method for enhancing personal hygiene and public health in a setting for trying on or determining the fit of one or more garments; the method comprising the steps of
obtaining one or more protective coverings according to claim 2;
applying the protective coverings in a setting for trying on or determining the fit of one or more garments;
maintaining the protective coverings in place during the process of trying on or determining the fit of one or more garments;
removing the protective coverings thereafter, and
disposing of the protective coverings.

14. The method of claim 13, wherein the setting is a retail apparel store.

15. The method of claim 13, wherein the protective coverings are commercially sterile.

16. The method of claim 13, wherein the step of disposing the protective coverings is done in a sanitary manner.

17. The method of claim 13, wherein the setting is a retail apparel store that sells bras, bathing suits, halter tops, lingerie, pajamas, sleepwear, robes, or apparel that is likely to come into direct contact with a woman's breasts.

18. The method of claim 13, wherein the protective coverings are provided in pairs.

19. A kit for improving public health in a retail apparel setting said kit comprising:
a) protective coverings according to claim 2;
b) instructions for the use, wearing, applying, and/or disposal of the protective coverings; and
c) a CD, DVD, or links to one or more Internet locations containing additional instructional material including but not limited a manual for use or video instruction for using the protective coverings for public health and/or personal hygiene.

20. The kit of claim 19 further comprising one or more of:
a) a dispenser for use in the fitting areas; said dispenser for dispensing individually or in pairs, the protective coverings to shoppers trying on or determining fit of a garment; and
b) one or more signs for use in the retail apparel store providing information in the public health risks associated with unprotected trying on or fitting such garments, or the public health benefits of using the protective coverings.
